(19) United States
(12) Patent Application Publication
(10) Pub. No.: US 2025/0134777 A1
(43) Pub. Date: May 1, 2025

(54) System and Method for Detecting the State of a Medical Device

(71) Applicant: Medcaptain Medical Technology Co., Ltd., Shenzhen (CN)

(72) Inventors: Minzhi LI, Shenzhen (CN); Jinbo YU, Shenzhen (CN); Xiang LI, Shenzhen (CN); Weixin DAI, Shenzhen (CN); Wenhao HUANG, Shenzhen (CN); Huangbin CHEN, Shenzhen (CN)

(73) Assignee: Medcaptain Medical Technology Co., Ltd., Shenzhen (CN)

(21) Appl. No.: 18/902,767

(22) Filed: Sep. 30, 2024

(30) Foreign Application Priority Data
Oct. 26, 2023 (CN) 202311407704.6

(51) Int. Cl. A61J 15/00

(52) U.S. Cl. CPC A61J 15/0076; A61J 15/0088

(57) ABSTRACT

A system and method are provided to detect the operational state of a medical device, which includes a controller, a drive member, and a drive control member, where the controller is configured to control the drive control member to drive the drive member to operate. The method includes: acquiring an operating state parameter of the drive member, and determining the state information of the medical device according to the operating state parameter of the drive member and a predetermined judgment rule. Therefore, the state information of the medical device can be determined, and state monitoring of the medical device is realized, thereby ensuring the reliability of the medical devices.

### REFERENCE TO RELATED APPLICATIONS

The invention claims priority of Chinese Patent Application No. 202311407704.6, entitled “Detection Method, Medical Device, and Computer-Readable Storage Medium” filed with the China National Intellectual Property Administration on Oct. 26, 2023, which is incorporated herein by reference in its entirety.

### TECHNICAL FIELD

This invention generally relates to medical devices, and more particularly, to a detection method, medical device, and computer-readable storage medium.

### BACKGROUND ART

At present, with the rapid development of the field of intelligent terminals, the field of medical equipment is also developing rapidly. For example, medical devices such as infusion pumps and syringe pumps can automatically inject nutrient fluids or drugs into patients and monitor the treatment of patients, thereby improving the efficiency and flexibility of clinical drug administration operations, and reducing the workload of nursing.

Through the use of state detection in medical devices such as infusion pumps and syringe pumps, it is possible to determine whether an abnormality occurs in the medical device. This invention seeks to provide a detection method of the state of a medical device.

### SUMMARY OF THE INVENTION

Some embodiments of the invention provide a detection method, a medical device, and a computer-readable storage medium, which can realize state detection of the medical device.

Some embodiments of the invention provide a detection method applied to a medical device, which comprises a controller, a drive member, and a drive control member, in which the controller is configured to control the drive control member to drive the drive member to operate. The method includes:
acquiring an operating state parameter of the drive member; and
determining state information of the medical device according to the operating state parameter of the drive member and a predetermined judgment rule.

In some embodiments, the operating state parameter is related to a load driven by the drive member.

In some embodiments, the operating state parameter comprises at least one of the following: a voltage value, a current value, a torque, and a rotation speed.

In some embodiments, the method further comprises outputting state information.

In some embodiments, the medical device further comprises a force sensor, the force sensor being placed at a load end to which the drive member is connected. The step of acquiring an operating state parameter of the drive member comprises acquiring a torque measurement voltage from the force sensor; and obtaining a torque value of the drive member according to a torque measurement voltage and a preset torque conversion factor.

In some embodiments, the step of obtaining the torque value of the drive member according to the torque measurement voltage and the preset torque conversion factor comprises determining a product of the torque measurement voltage and the predetermined torque conversion factor as the torque value of the drive member.

In some embodiments, the medical device is a nutrient infusion device, and the nutrient infusion device further comprises a speed reducer (such as reduction gear(s)) and a roller structure; the drive member is drivingly connected to the speed reducer, and the speed reducer is drivingly connected to the roller structure; wherein the roller structure is configured to rotate under the drive of the drive member and to drive the flow of a fluid in consumables; and the roller structure is removably connected to the speed reducer by a quick-release coupling clasp.

In some embodiments, the step of determining the state information of the medical device according to the operating state parameters of the drive member and the predetermined judgment rule comprises:
comparing an operating current of the drive member with a range of consumable dislodgement current corresponding to the rotation speed of the drive member during a period of use of the consumables;
outputting an alarm message for shedding of the consumables when the operating current of the drive member is within the range of the consumable dislodgement current.

In some embodiments, the nutrient solution infusion apparatus further comprises an in-position detection member, the in-position detection member being disposed opposite the roller structure;
the step of outputting the alarm message indicating that the consumable has become dislodged (below: “dislodgement”) when the operating current of the drive member is within the range of the consumable dislodgement current comprises:
outputting the alarm message indicating dislodgement of the consumable when the operating current of the drive member is within the range of the consumable dislodgement current and the in-position detection member detects that the roller structure is in position.

In some embodiments, the method further comprises:
outputting an exception message indicating that the roller structure is in an incorrect position when the operating current of the drive member is within the range of the consumable dislodgement current and the in-position detection member detects that the roller structure is not in position.

Some embodiments of the invention provide a detection device comprising:
an acquisition module configured to acquire an operating state parameter of a drive member in a medical device; and
a processing module configured to determine state information of the medical device according to the operating state parameter of the drive member and a predetermined judgment rule.

Some embodiments of the invention provide a medical device, comprising: a controller, a drive member, a drive control member; wherein the controller is configured to control the drive control member to drive the drive member to operate, and further configured to implement the detection method involved in the above embodiments.

Some embodiments of the invention provide a computer-readable storage medium having computer instructions stored therein, wherein the computer instructions when executed by a processor are configured to realize the detection method involved in the above embodiments when executed by a processor.

According to the detection method, for the medical device, and the computer-readable storage medium provided by the invention, detection of the operation state of the drive member is realized by acquiring the operating state parameters of the drive member, and then the state information of the medical device is determined according to the operating state parameters of the drive member and predetermined judgment rules, so that the state information of the medical device can be determined and the state monitoring of the medical device can be realized, thereby ensuring the reliability of the medical device during operation.

### BRIEF DESCRIPTION OF THE DRAWINGS

The accompanying drawings herein, which are incorporated into and form a part of the specification, illustrate embodiments consistent with the invention and are used in conjunction with the specification to explain the principles of the invention.

FIG. 1 shows a flowchart of a detection method provided by some embodiments of the invention.

FIG. 2 shows a schematic circuit diagram of a drive control member provided by some embodiments of the invention.

FIG. 3 shows a flowchart of another detection method provided by some embodiments of the invention.

FIG. 4 shows a structural schematic diagram of a nutrient infusion device.

FIG. 5 shows a schematic diagram of a consumable being mounted on a consumable support plate.

FIG. 6 shows a schematic diagram of the consumable being mounted in the nutrient infusion device by the consumable support plate.

The reference signs are as follows:
100, controller; 200, drive control member; 300, drive member; 500, roller structure; 601, consumable; 603, consumable support plate; 700, acquisition circuit; 701, bridge arm amplifier; Rref, bridge arm reference resistor; 401, Hall sensor; 402, magnet.

Various embodiments of the invention are shown by means of the above-described accompanying drawings, which will be described in greater detail later. These accompanying drawings and textual descriptions are not intended to limit the scope of the invention idea in any way, but rather to illustrate the concepts of the invention for those skilled in the art by reference to particular embodiments.

### DETAILED DESCRIPTION

Examples of embodiments will be described herein in detail, which are represented in the accompanying drawings. When the following description relates to the accompanying drawings, the same numerals in the different accompanying drawings indicate the same or similar elements unless otherwise indicated. The embodiments described in the following examples of embodiments do not represent all embodiments consistent with the invention; rather, they are only examples of devices and methods consistent with some aspects of this invention as detailed in the appended claims.

It should be noted that the user information (including, but not limited to, user device information, user personal information) and data (including, but not limited to, data used for analysis, stored data, displayed data) involved in the invention are information and data authorized by the user or fully authorized by the parties, and that the collection, use, and processing of the relevant data are required to comply with relevant laws, regulations, and standards of the relevant countries and regions, and are provided with corresponding operational portal for the user to choose to authorize or refuse.

State detection for medical devices such as infusion pumps and injection pumps can determine whether an abnormality occurs in the medical device. The invention is intended to provide a method for detecting the state of a medical device.

### Embodiment 1

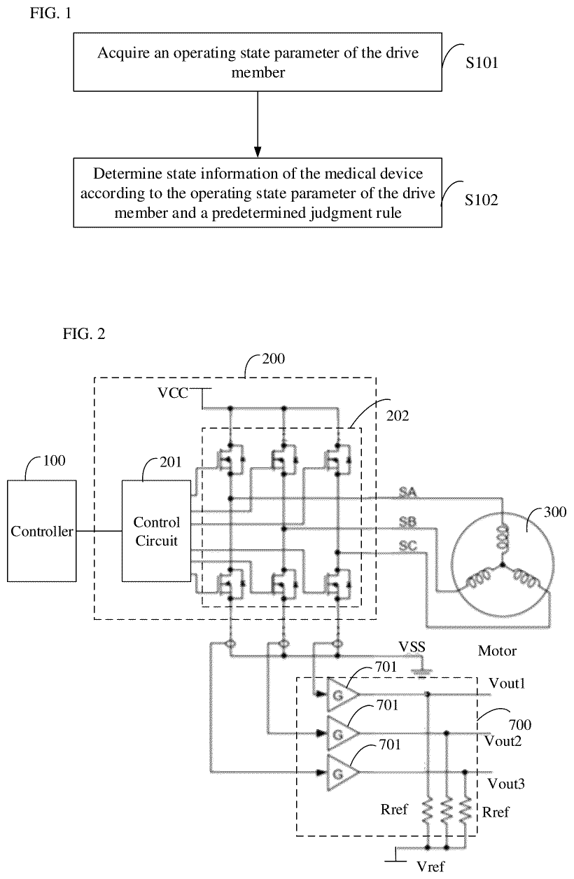

As shown in FIG. 1, some embodiments of this invention provide a detection method for use with a medical device. The medical device includes a drive member, a drive control member, and a controller, which is configured to control the drive control member to drive the drive member to operate. This method includes the following steps.

S101, an operating state parameter of the drive member is acquired.

The operating state parameter of the drive member characterizes the operating state of the drive member.

In some embodiments, the operating state parameter includes at least one of the following: a voltage value, a current value, a torque, and a rotation speed.

S102, the state information of the medical device is determined according to the operating state parameter of the drive member and the predetermined judgment rule.

The predetermined judgment rule is selected to determine the state information of the medical device. The state information of the medical device is determined by inputting the operating state parameter of the drive member as a parameter of the the predetermined judgment rule and determining a judgment result output according to the predetermined judgment rule.

In some embodiments, the predetermined judgment rule includes at least one judgment range. If the operating state parameter of the drive member lies within one of the predetermined judgment ranges, it is determined that the medical device is in a corresponding state.

In some embodiments, the predetermined judgment rule includes a judgment formula and the at least one judgment range. An intermediate result is obtained by inputting the operating state parameter of the drive member into the judgment formula for calculation. When the intermediate result lies within one of the predetermined judgment ranges, it is determined that the medical device is in the corresponding state.

In some embodiments, the predetermined judgment rule includes a predetermined application scenario and the at least one judgment range. As it is possible for the operating state parameters of the drive member to overlap and intersect the judgment ranges in different application scenarios, it is easy to cause the medical device to be unable to distinguish the corresponding state information. Therefore, it is possible to set an application scenario in the medical device by the user in advance, and according to this application scenario, when the operating state parameter of the drive member is located in one of the judgment ranges, the corresponding operating state of the medical device under this application scenario is determined. By adding the conditions of the predetermined application scenario, the accuracy of the medical device in determining the state information of the drive member is improved.

In the above technical solution, by acquiring the operating state parameters of the drive member, the detection of the operating state of the drive member is realized, and then according to the operating state parameters of the drive member and the predetermined judgment rules, the state information of the medical device is determined, so that the state information of the medical device can be determined, and the state monitoring of the medical device can be realized. In this way, the reliability of the medical device can be ensured in the process of using the medical device.

### Embodiment 2

Some embodiments of the invention provide a detection method that is applied to a medical device. This method includes the following steps.

S201, an operating state parameter of the drive member is acquired.

The operating state parameter is related to a load driven by the drive member. That is, the operating state parameter of the drive member characterizes the operational state of the drive member related to the load when the drive member drives the load.

S202, the state information of the medical device is determined according to the operating state parameter of the drive member and the predetermined judgment rule.

Since the operating state parameter of the drive member is related to the load driven by the drive member, the judgment result outputted by the predetermined judgment rule is also a judgment result related to the drive member when driving the load, so as to determine the state information of the medical device when driving the load.

In the above technical solution, by acquiring the operating state parameters of the drive member related to the load, the detection of the operating state of the drive member when driving the load is realized, and then according to the operating state parameters of the drive member and the predetermined judgment rule, the state information of the medical device when driving the load is determined, so as to realize the monitoring of the state of the medical device when driving the load, and thus ensure the reliability of the medical device in the process of using the medical device.

### Embodiment 3

Some embodiments of the invention provide a detection method, which is applied to a medical device. This method includes the following steps.

S301, an operating state parameter of the drive member is acquired.

The operating state parameter includes a working current of the drive member. A voltage sensor may be used to detect the working voltage of the drive member and output a voltage value, which may be converted and output the working current of the drive member. A current sensor may also be used to output the detected working current of the drive member.

S302, according to the operating state parameter of the drive member and the predetermined judgment rule, the state information of the medical device is determined.

The predetermined judgment rule includes a preset current range. When the operating current of the drive member lies within the preset current range, the operating state of the drive member is in a normal state. When the operating current of the drive member lies outside the preset current range, the operating state of the drive member is in an abnormal state.

The operating current of the drive member can characterize the load connected to the drive member. When the connected load is normal, the operating current of the drive member is within the preset current range. When the connected load is abnormal, the operating current of the drive member is outside the preset current range.

When the operating current of the drive member is greater than the upper limit of the preset current range, this indicates that the drive member is generating a relatively large driving force, such that there may be additional resistance hindering the drive member. When the operating current of the drive member is less than the lower limit value of the predetermined current range, this indicates that the drive member output driving force is relatively small, which may indicate that there may be a load that is not properly connected to the load end of the drive member.

The preset current range is also related to the type of load and the rotation speed of the drive member. Different load types correspond to different preset current ranges.

In the case where the load type does not change, the higher the rotation speed, the higher the operating current corresponding to the normal driving of the load will be, which corresponds to the lower and upper limit values of the preset current range.

For example, when the rotation speed of the drive member is at 60 rpm, the corresponding operating current might be in the range 100 mA˜110 mA. When the rotation speed of the drive member is at 70 rpm, the corresponding operating current might then be in the range 120 mA˜130 mA.

In the above technical solution, by detecting the operating current of the drive member and determining the state information of the medical device when driving the load according to the operating current of the drive member and the preset current range, the operating state of the medical device when carrying the load can be determined, and the reliability of the medical device in the process of using the medical device can be ensured in this way. In addition, the detection of the working state of the medical device when carrying a load can be realized without additional mechanical sensors, which greatly simplifies the complexity of the mechanical structure design.

### Embodiment 4

Some embodiments of the invention provide a detection method, which is applied to a medical device. This method includes the following steps.

S401, an operating state parameter of the drive member is acquired.

The operating state parameter includes a torque value.

The medical device further includes a force sensor. The force sensor is placed at a load end connected to the drive member, and the force sensor is configured to detect a torque at the load end connected to the drive member.

In one embodiment, the step of acquiring the operating state parameter of the drive member includes the following steps.

A torque measurement voltage is acquired from the force sensor, and a torque value of the drive member is obtained according to the torque measurement voltage and a preset torque conversion factor. Thus, the torque value of the drive member is obtained from the force sensor.

The predetermined torque conversion coefficient is determined according to an internal structure of the force sensor, which may be determined by querying an operating parameter of the force sensor.

In some embodiments, the step of obtaining the torque value of the drive member according to the torque measurement voltage and the preset torque conversion factor includes the following step.

A product of the torque measurement voltage and the predetermined torque conversion factor is determined as a torque value of the drive member.

In some embodiments, a current sensor may be configured to output a voltage value after detecting the working current of the drive member, and to output the working current of the drive member after converting the voltage value, and then to obtain the torque value of the drive member as a function of the working current of the drive member and the preset current torque conversion coefficient. For example, the current torque conversion factor may be 0.9.

S402, the state information of the medical device is determined according to the operating state parameter of the drive member and the predetermined judgment rule.

The predetermined judgment rule includes a preset torque range. When the torque value of the drive member lies within the preset torque range, the operation state of the drive member is in a normal state. When the torque value of the drive member lies outside the preset torque range, the operation state of the drive member is in an abnormal state.

When the torque value of the drive member is greater than an upper limit value of the predetermined torque range, there may be additional resistance hindering the drive member. When the torque value of the drive member is less than the lower limit value of the predetermined torque range, then the load may not be properly connected to the load end of the drive member.

The preset torque range will typically also be related to the type of load, such that different load types correspond to different preset torque ranges. The preset torque range may be set according to the load type, so as to adapt to the detection of the working state when carrying different types of loads, thereby improving the accuracy of the detection results.

In the above technical solution, by detecting the torque value of the drive member, and determining the state information of the medical device when driving the load according to the torque value of the drive member and the preset torque range, it is possible to determine the working state of the medical device when carrying the load, so as to ensure the reliability of the medical device in the process of using the medical device.

### Embodiment 5

Some embodiments of the invention provide a detection method, which is applied to a medical device. The method includes the following steps.

S501, an operating state parameter of the drive member is acquired.

The operating state parameter includes a voltage value. A current sensor may be used to detect the operating current of the drive member and convert the current value to a voltage value.

S502, according to the operating state parameter of the drive member and the predetermined judgment rules, the state information of the medical device is determined.

The predetermined judgment rule includes a preset voltage range. When the voltage value of the drive member lies within the preset voltage range, the operation state of the drive member is in a normal state. When the voltage value of the drive member lies outside the preset voltage range, the operation state of the drive member is in an abnormal state.

The operating voltage of the drive member may characterize the load condition to which the drive member is connected, and when the connected load is normal, the operating voltage of the drive member will lies within the preset voltage range. When the connected load is abnormal, the operating voltage of the drive member lies outside the preset voltage range.

When the operating voltage of the drive member is greater than the upper limit of the preset voltage range, this may indicate that the drive member outputs a relatively large driving force, there may be additional resistance hindering the drive member. When the operating voltage of the drive member is less than the lower limit value of the predetermined voltage range, this may indicate that the output driving force of the drive member is relatively small and that there may be a load that is not properly connected to the load end of the drive member.

In the above-described technical solution, by detecting the voltage value of the drive member, and determining the state information of the medical device when driving the load according to the voltage value of the drive member and the preset voltage range, the working state of the medical device when carrying the load can be determined, and the reliability of the medical device in the process of using the medical device can be ensured. The dynamic detection of the load can be realized without additional mechanical sensors, thereby greatly simplifying the complexity of mechanical structure design.

FIG. 2 shows a circuit schematic diagram of the drive control member 200 according to some embodiments of the invention. The drive control member 200 includes a drive circuit 202 and a control circuit 201. An output end of the drive circuit 202 is connected to an electrical end of the drive member 300, and an input end of the drive circuit 202 is connected to an output end of the control circuit 201. The control circuit 201 is configured to output a control signal. The control signal controls the drive circuit 202 to output a drive current, and the drive current drives the drive member 300 to operate.

The controller 100 is connected to the control circuit 201, and the controller 100 outputs control instructions. The control circuit 201 is configured to convert the control instructions into control signals for output.

In some embodiments, as shown in FIG. 2, when the drive member 300 is a motor, the drive circuit 202 is a three-phase bridge arm circuit, and the control circuit 201 is configured to output a Pulse Width Modulated (PWM) signal.

In some embodiments, as shown in FIG. 2, the medical device further includes an acquisition circuit 700, which is configured to acquire an operating current of the drive member 300, so that the medical device determines an operating state of the medical device according to the operating current of the drive member and the predetermined judgment rule.

In some embodiments, as shown in FIG. 2, when the driving circuit 202 is a three-phase bridge arm circuit, the acquisition circuit 700 includes three bridge arm amplifiers 701 and three bridge arm reference resistors R_{ref}. An input end of each bridge arm amplifier 701 is connected to the corresponding bridge arm for amplifying the bridge arm current and then outputting the bridge arm current. The first end of each bridge arm reference resistor R_{ref} is connected to the output end of the corresponding bridge arm amplifier 701, and the second end of the bridge arm reference resistor R_{ref} is connected to a reference voltage V_{ref}. By reading the voltage at the output end of the bridge arm amplifier 701, it is realized to obtain a voltage value, which characterizes the operating state of the motor.

In some embodiments, the acquisition circuit 700 further includes a conversion module (not shown in the figures). The conversion module is configured to receive the voltage on the bridge arm reference resistor R_{ref} and convert the voltage on the bridge arm reference resistor R_{ref} to the operating current of the motor.

The working current of the motor can be calculated according to the following formula:

$$I = \frac{( {V}_{out} - {V}_{ref} ) \times G}{{R}_{ref}} ;$$

Here, I denotes the current value of the motor, Vₒᵤₜ denotes the output voltage of the bridge arm amplifier 701, V_{ref} denotes the reference voltage, G denotes the amplification gain of the bridge arm amplifier 701, and R_{ref} denotes the bridge arm reference resistance.

Vₒᵤₜ may be the output terminal voltage Vₒᵤₜ₁ of the first bridge arm amplifier, but may also be the output terminal voltage Vₒᵤₜ₂ of the second bridge arm amplifier, or the output terminal voltage Vₒᵤₜ₃ of the third bridge arm amplifier, or the average value of the output terminal voltages of any two of the three bridge arm amplifiers, of even the average value of the output terminal voltages of the three bridge arm amplifiers.

In some embodiments, the acquisition circuit 700 also includes a filtering circuit (not shown), which is connected to the input end of the bridge arm amplifier 701. The bridge arm current is then filtered by the filtering circuit and output to the bridge arm amplifier 701, so that noise signals are filtered out of the bridge arm current and the bridge arm current can be more accurately determined. The bridge arm current can be amplified and then output, and then the amplified current can be sampled. The sampled voltage value may be obtained as the operating state parameter of the drive member, thereby improving accuracy.

### Embodiment 6

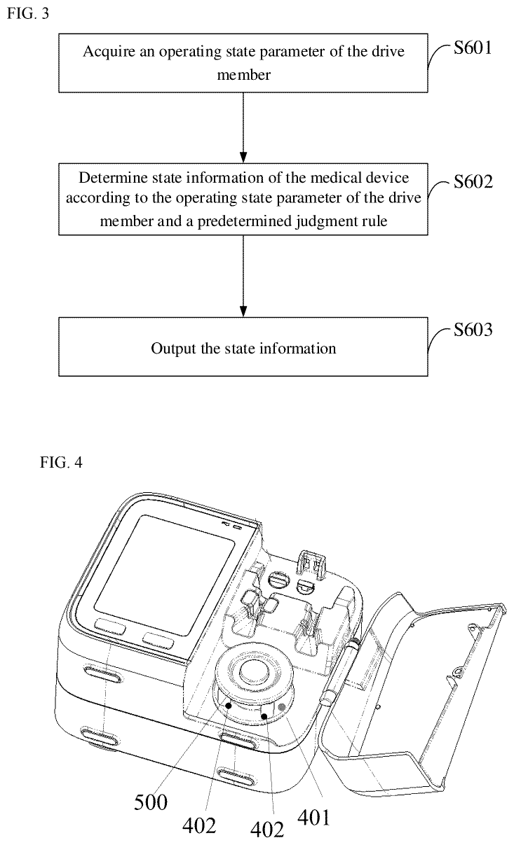

As shown in FIG. 3, some embodiments of the invention provide a detection method which is applied to a medical device. This method includes the following steps.

S601, an operating state parameter of the drive member is acquired.

The operating state parameter of the drive member characterizes the operation state of the drive member, and the operating state parameter is related to a load driven by the drive member.

S602, the state information of the medical device is determined according to the operating state parameter of the drive member and the predetermined judgment rule.

In some embodiments, the predetermined judgment rule includes at least one judgment range. If the operating state parameter of the drive member lies within one of the predetermined judgment ranges, the medical device is determined to be in a corresponding state.

In some embodiments, the predetermined judgment rule includes a judgment formula and the at least one judgment range. An intermediate result is obtained by substituting the operating state parameter of the drive member into the judgment formula for calculation. When the intermediate result lies within one of the predetermined judgment ranges, this is taken to indicate that the medical device is in the corresponding state.

In some embodiments, different types of loads require different driving forces, so a plurality of judgment ranges may be determined in advance, with each judgment range corresponding to a type of load. Thus, the type of load carried on the medical device is determined according to the operating state parameter of the drive member.

In some embodiments, where different states of the load require different driving forces, a plurality of judgment ranges may be determined in advance, with each judgment range corresponding to one state of the load. The state of the load carried on the medical device is thus determined according to the operating state parameters of the drive member.

S603, the state information is output.

After determining the state information of the medical device, the state information is output.

In some embodiments, the medical device is provided with a display screen, and the state information is displayed on the display screen.

In some embodiments, the medical device is provided with a speaker to output the state information by voice announcement.

In some embodiments, the output state information may include load state information, e.g., a load abnormal state or a load normal state.

In some embodiments, the output state information may include a load type carried.

In some embodiments, when a load type is detected, if the load type is not a required load type, information that the load type is abnormal may be output. If the load type carried is the requested load type, information that the load type is normal may be output.

In the above-described technical solution, by acquiring the operating state parameters related to the load in the drive member, the detection of the operating state of the drive member when driving the load is realized. Then, according to the operating state parameter of the drive member and the predetermined judgment rule, the state information of the medical device when driving the load is determined. The monitoring of the state of the medical device when driving the load is realized, and the corresponding state information is output, so as to realize the detection of a variety of states.

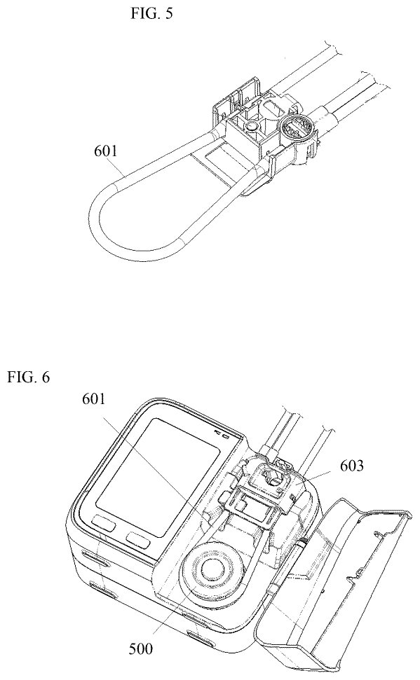

In some embodiments, the medical device is a nutrient infusion device, and FIG. 4 shows a structural schematic diagram of an example of such a nutrient infusion device. FIG. 5 shows a schematic diagram of a consumable 601 being mounted on a consumable support plate 603, and FIG. 6 shows a schematic diagram of the consumable 601 being fixed to the nutrient infusion device by the consumable support plate 603. The nutrient infusion device further includes a speed reducer (not shown in the figures) and a roller structure 500, the drive member is drivingly connected to the speed reducer, and the speed reducer is drivingly connected to the roller structure 500.

The roller structure 500 is configured to rotate under the drive force of the drive member 300 and to drive fluid flow in the consumable 601. The roller structure 500 is removably connected to the speed reducer by a quick release coupling clasp.

The consumable 601 is mounted on the roller structure 500 such that the roller structure 500 can “squeeze” and drive the fluid in the consumable 601 as it rotates. The rotation speed of the roller structure 500 is related to the fluid flow rate, in particular, the fluid flow rate is the product of the rotation speed of the roller structure 500 and a conversion factor between the rotation speed and the flow rate.

The consumable 601 may be a silicone tube that is mounted in the roller structure 500 and then secured to the body of the nutrient infusion device by the consumable support plate 603.

### Embodiment 7

Some embodiments of the invention provide a detection method, which is applied to a nutrient solution infusion device. This method includes the following steps.

S701, an operating state parameter of the drive member is acquired.

The operating state parameter of the drive member includes an operating current of the drive member, which is measured when the medical device is running with a load.

For example, the working current of the drive member may be measured when the nutrient infusion device is running and when a roller structure is installed and consumables are installed in the roller structure.

S702: the state information of the medical device is determined according to the operating state parameter of the drive member and the predetermined judgment rule.

The predetermined judgment rule includes a “consumable in-position” current range and/or a “consumable dislodgement” current range.

When the consumable is mounted within the roller structure, a plurality of consumable in-position currents are obtained by allowing the nutrient infusion device to perform a plurality of tests at a certain test flow rate, and the plurality of consumable in-position currents are statistically analyzed to obtain the consumable in-position current range.

A plurality of consumable dislodgement currents is obtained by performing a plurality of tests of the nutrient infusion device when the consumable is not located in the roller structure, and the plurality of consumable dislodgement currents are statistically analyzed to obtain the consumable dislodgement current range.

For example, when a standard consumable is mounted on the roller structure of a nutrient infusion device, infusion flow testing might be carried out with a flow rate of 700 mL/h to obtain test currents for multiple motors. The average value of the test current of multiple motors might be calculated as 125 mA, so that the consumable in-position current range could be set as (125 mA, 125 mA+12.5 mA).

For example, with a standard consumable mounted on a roller structure, the nutrient infusion device could be allowed to perform a fluid infusion test at a flow rate of 1200 mL/h, so as to obtain test currents of the plurality of motors. The average value of the test currents of the plurality of motors might then be calculated to be 200 mA, so the consumable in-position current range could be set to be (200 mA, 220 mA).

In some embodiments, the operating current of the drive member is compared with the consumable in-position current range of the drive member during a period of use of the consumables. When the operating current of the drive member is not in the consumable in-position current range, an alarm message of dislodgement of the consumable is output. When the operating current of the drive member is within the consumable in-position current range, the consumable-in-position information is output.

In some embodiments, when the operating current of the drive member is less than a lower limit of the consumable in-position current range, the alarm message indicating dislodgement of the consumable is output.

In some embodiments, the operating current of the drive member is compared to the consumable dislodgement current range of the drive member during a period of use of the consumables. When the operating current of the drive member is within the consumable dislodgement current range, the alarm message for shedding of the consumable is output. When the operating current of the drive member is not within the consumable dislodgement current range, the consumable in-position message is output.

In some embodiments, the operating current of the drive member is compared to the consumable in-position current range during a period of use of the consumables. When the operating current of the drive member lies in the consumable in-position current range, the consumable in-position message is output. When the operating current of the drive member is not in the consumable in-position current range, the operating current of the drive member is compared with the consumable dislodgement current range of the drive member, and when the operating current of the drive member lies in the consumable dislodgement current range, the alarm message for dislodgement of the consumable is output.

In the above technical solution, if there is a great difference between the current of the drive member when the nutrient infusion pump is running with load and the current of the drive member when it is running with no load, the working current of the drive member may be determined and compared with the consumable in-position current range. Whether or not the consumables are located in the roller structure is also determined, thus realizing the state detection of whether the consumables in the nutrient infusion device are located in the roller structure without adding additional mechanical structures, simplifying the structure of the nutrient infusion pump.

### Embodiment 8

Some embodiments of the invention provide a detection method, which is applied to a medical device. The method specifically includes the following steps.

S801, an operating state parameter of the drive member is acquired.

The operating state parameter of the drive member includes an operating current of the drive member when the medical device is running.

For example, the working current of the drive member when the nutrient infusion device is running when a roller structure is installed and consumables are installed in the roller structure is measured.

S802, the state information of the medical device is determined according to the operating state parameter of the drive member and the predetermined judgment rule.

The predetermined judgment rule includes a consumable in-position current range. A plurality of consumable in-position currents are determined by performing a plurality of tests of the nutrient infusion device when the consumable is disposed in the roller structure, and the plurality of consumable in-position currents is statistically analyzed to obtain the consumable in-position current range.

The medical device further includes an in-position detection member. The in-position detection member is disposed opposite the roller structure, and is configured to detect whether the roller structure is mounted on the drive member.

In some embodiments, the step of determining the state information of the medical device according to the operating state parameters of the drive member and the predetermined judgment rule includes the following steps.

Step a1, an operating current of the drive member is compared with a consumable in-position current range of the drive member.

Step b1, when the operating current of the drive member is not in the consumable in-position current range, the detection result of the in-position detection member is continuously acquired.

Step c1, when the in-position detection member detects that the roller structure is in position, an alarm message of the dislodgement of the consumables is output.

Step d1, when the in-position detection member detects that the roller structure is not in position, the roller structure in-position abnormality information is output.

Step e1, when the operating current of the drive member is within the consumable in-position current range, the consumable in-position information is output.

In some embodiments, the state information of the medical device is determined according to the operating state parameters of the drive member and the predetermined judgment rules, including the following steps.

Step a2, the operating current of the drive member is compared with the consumable dislodgement current range of the drive member.

Step b2, when the operating current of the drive member is within the consumable dislodgement current range, the detection result of the dislodgement detection member. is acquired continually

Step c2, when the dislodgement detection member detects that the roller structure is in position, the alarm message indicating dislodgement of the consumable is outputted.

Step d2, when the dislodgement detection member detects that the roller structure is not in position, the roller structure dislodgement abnormality information is output.

Step e2, when the operating current of the drive member is not within the consumable dislodgement current range, the consumable in-position information is output.

In some embodiments, the in-position detection member includes a Hall sensor 401 and a plurality of magnets 402. The Hall sensor 401 is disposed inside the nutrient infusion device and is arranged close to the roller structure. The plurality of magnets 402 are provided on the roller structure arranged along a circumferential direction of the roller structure.

When the drive member of the nutrient infusion device drives the wheel structure to rotate, each magnet 402 moves close to the Hall sensor 401 in turn, and then moves far away from the Hall sensor 401. When the Hall sensor 401 moves close to the magnet 402, the Hall sensor 401 outputs a signal with a strength that becomes larger. When the Hall sensor 401 is far away from the magnet 402, the Hall sensor 401 outputs a signal with a strength that becomes smaller, such that the Hall sensor 401 outputs a pulse signal. The number of pulsed signals output by the Hall sensor 401 during one full rotation of the roller structure is related to the number of magnets 402.

When the wheel structure is not mounted on the drive member, the Hall sensor 401 output signal does not change because there is no magnet 402 to influence it.

By acquiring the output signal of the Hall sensor 401, whether the Hall sensor 401 outputs a pulse signal can be determined. If the pulse signal is output, this indicates that the roller structure is mounted on the drive member. If a signal with a constant high level or a low level is output, this indicates that the roller structure is not mounted on the drive member.

The step of determining the state information of the medical device according to the operating state parameters of the drive member and the predetermined judgment rule specifically includes the following steps.

Step a2, the operating current of the drive member is compared with a consumable in-position current range of the drive member.

Step b2, when the operating current of the drive member is not within the consumable in-position current range, the detection result of the Hall sensor 401 is acquired continually.

Step c2, when the Hall sensor 401 outputs a pulse signal, an alarm message indicating dislodgement of the consumables is output.

Step d2, when the Hall sensor 401 outputs a signal with a high level or a low level, the roller structure in position abnormality information is output.

Step e2, when the operating current of the drive member is within the consumable in-position current range, the consumable in-position information is output.

By setting the Hall sensor 401 on the nutrient infusion device and the magnets 402 on the roller structure, the roller structure will rotate under the drive of the drive member when it is mounted on the drive member, causing the Hall sensor 401 to output a pulse signal, which may be taken as an indication that the roller structure is in position.

In the above technical solution, if the roller structure is not mounted on the drive member and the consumables fall off from the roller structure will cause the drive member to rotate with no load, so that the operating current of the drive member does not lie within the consumable in-position current range. In order to improve the accuracy of the detection results, the operating current of the drive member may be compared with the consumable in-position current range. When the operating current of the drive member does not lie within the consumable in-position current range, whether the roller structure is mounted to the drive member is determined continuously. When the roller structure is mounted on the drive member, this may be taken as indication that the consumable in the medical device is not located in the roller structure; no mechanical structures necessary, simplifying the structure of the nutrient infusion pump.

### Embodiment 9

Some embodiments of the invention provide a detection method which is applied to a nutrient solution infusion device. This method includes the following steps.

S901, an operating state parameter of the drive member is acquired.

The operating state parameter of the drive member includes a torque value of the drive member. The torque of the drive member when the medical device is running with a load is measured.

For example, the torque value of the drive member when the nutrient infusion device is running with a roller structure installed and consumables are installed in the roller structure may be, measured.

S902: the state information of the medical device is determined according to the operating state parameter of the drive member and the predetermined judgment rule.

The predetermined judgment rule includes a range of consumable in-position torque values. When the consumable is located in the roller structure, a plurality of consumable in-position torque values is obtained by allowing the nutrient infusion device to perform multiple tests at a certain test flow rate, and the plurality of consumable in-position torque values is statistically analyzed to obtain the consumable in-position torque value range.

As one example, if the standard consumables are installed on the roller structure, and the liquid infusion tests are performed on the nutrient infusion device at a flow rate of 700 mL/h, the test torque values of multiple motors can be measured. The average value of the test torque values of multiple motors might for example be calculated to be 112.5 mN·m (112.5 millinewton meters) and the consumable in-position torque value range could be set to be (112.5 mN·m, 112.5×1.1 mN·m).

For example, assume that a standard consumable is mounted on a roller structure, and the nutrient infusion device is allowed to perform a liquid infusion test at a flow rate of 1200 mL/h. The test torque values of a plurality of motors may then be measured, and the average value of the test torque values of the plurality of motors might be calculated to be 180 mN·m, and the consumable in-position torque value range is set to be (180 mN·m, 180 mN·m+18 mN·m).

During the use period of the consumable, the torque value of the drive member is compared with the consumable in-position torque value range of the drive member. When the torque value of the drive member is not within the consumable in-position torque value range, an alarm message indicating dislodgement of the consumable is output. When the torque value of the drive member is within the consumable in-position torque value range, the consumable in-position information is output.

In some embodiments, when the torque value of the drive member is less than a lower limit value of the consumable in-position torque value range, the alarm message indicating dislodgement of the consumable is output.

In the above technical solution, since there is a great difference between the torque value of the driving member when the nutrient infusion pump is running with load and the torque value of the driving member when it is running with no load, acquiring the torque value of the driving member, comparing the torque value of the driving member with the range of in-situ torque values of the consumables, and therefrom determining whether or not the consumables are disposed in the roller structure, realizes state detection of whether or not the consumables in the nutrient infusion device are disposed in the roller structure.

### Embodiment 10

Some embodiments of the invention provide a detection method, which is applied to a medical device. This method comprises the following steps.

S1001, an operating state parameter of the drive member is acquired.

The operating state parameter of the drive member includes an operating current of the drive member. The operating current of the drive member when the medical device is running with a load is acquired.

For example, the operating current of the drive member when the nutrient infusion device is running with a roller structure installed and consumables installed in the roller structure may be measured.

S1002, the state information of the medical device is determined according to the operating state parameters of the drive member and the predetermined judgment rule.

The predetermined judgment rule includes a consumable replacement current threshold. The consumable replacement current threshold characterizes the timing of replacing the consumable. If the consumable is used for a long period of time, the tension of the consumable will decay, and the decay of the tension of the consumable will reduce the precision of the infusion.

In some embodiments, a plurality of consumable replacement currents is acquired by performing multiple tests of the nutrient infusion device at a certain test flow rate with unused consumables mounted in the roller structure, and the plurality of consumable replacement currents are statistically analyzed to obtain the consumable replacement current threshold.

For example, a standard consumable may be mounted on the roller structure, and the nutrient infusion device be allowed to perform a test of infusing liquid at a flow rate of 700 mL/h, A plurality of test currents of the motors is acquired, and an average of the test currents of the plurality of motors might be calculated to be 125 mA, so the consumable replacement current threshold is set to be 125 mA+125×0.02 mA.

In some embodiments, the consumables are mounted in a roller structure. By testing the full life cycle of the consumables, the operating currents as well as the infusion accuracy of the drive member in the nutrient infusion device at different periods are acquired. The working current of the drive member corresponding to the time when the infusion precision of the nutrient infusion device is lower than a preset threshold is taken as the consumable replacement current threshold.

The step of determining the state information of the medical device according to the operating state parameter of the drive member and the predetermined judgment rule may include the following steps.

The operating current of the drive member is compared with the consumable replacement current threshold, and consumable replacement reminder information is output when the operating current of the drive member is less than the consumable replacement current threshold.

In the above technical solution, since the working current of the drive member also decreases when the tension force of the consumables decays, the working current of the drive member is detected and is compared with the consumables replacement current threshold. If the working current is less than the consumables replacement current threshold, it is determined that the tension force of the consumables decays, thereby reminding the user to replace the infusion consumables.

### Embodiment 11

Some embodiments of the invention provide a detection method which is applied to a medical device. This method comprises the following steps.

S1101, an operating state parameter of the drive member is acquired.

The operating state parameter of the drive member includes a torque value of the drive member. The torque value of the drive member when the medical device is running with a load is acquired.

For example, the torque value of the drive member when the nutrient infusion device is running with a roller structure installed and consumables installed in the roller structure may be acquired.

S1102, the state information of the medical device is acquired according to the operating state parameter of the drive member and the predetermined judgment rule.

The predetermined judgment rule includes a consumable replacement torque value threshold. The consumable replacement torque value threshold characterizes the timing of replacing the consumable. If the consumable is used for an extended period of time, the tension force of the consumable will decay, and the decaying tension force of the consumable will reduce the precision of the infusion.

In some embodiments, a plurality of consumable replacement torque values is obtained by performing a plurality of tests on a nutrient infusion device at a certain test flow rate with unused consumables mounted in a roller structure, and the plurality of consumable replacement torque values are statistically analyzed to obtain a consumable replacement torque value threshold.

For example, a standard consumable is mounted on the roller structure, and the nutrient infusion device is allowed to perform a test of infusing liquid at a flow rate of 700 mL/h, a plurality of motor test torque values are acquired. An average value of the plurality of motor test torque values is calculated to be 112.5 Nm, and setting a threshold of the torque value for consumable replacement to be 112.5 Nm+112.5×0.02 Nm.

In some embodiments, the consumables are installed in the roller structure, and the torque value of the drive member in the nutrient infusion device at different times, and the infusion precision of the nutrient infusion device at different times are obtained by testing the full life cycle of the consumables. The torque value of the drive member corresponding to the time when the infusion precision of the nutrient infusion device is lower than a preset threshold value is taken as the consumable replacement torque value threshold value.

The step of determining the state information of the medical device according to the operating state parameter of the drive member and the predetermined judgment rule may include the following steps.

The torque value of the drive member is compared with the consumable replacement torque value threshold, and the consumable replacement reminder information is output when the torque value of the drive member is less than the consumable replacement torque value threshold.

In the above technical solution, since the torque value of the drive member also decreases when the tension force of the consumables decays, the torque value of the drive member is detected and the torque value of the drive member is compared with the consumable replacement torque value threshold. If the torque value is less than the consumables replacement torque value threshold, it is determined that the tension force of the consumables decays, thereby reminding the user to replace the infusion consumables.

### Embodiment 12

Some embodiments of the invention provide a detection method, which is applied to a medical device. The method includes the following steps.

S1201, an operating state parameter of the drive member is acquired.

The operating state parameter of the drive member includes an operating current of the drive member when the medical device is running with a load is acquired.

For example, the working current of the drive member when the nutrient infusion device is running with a roller structure installed and consumables installed in the roller structure may be acquired.

S1202, the state information of the medical device is determined according to the operating state parameters of the drive member and the predetermined judgment rules.

The predetermined judgment rule includes standard current ranges of a plurality of types of consumables. The tension force of the consumables varies from one consumable type to another, and the corresponding operating current of the drive member is also different.

For each type of consumable, the standard current ranges of various types of consumables may be obtained by conducting multiple tests of the nutrient infusion device when each type of consumable is mounted in the roller structure.

For example, for type A consumables, by conducting multiple tests on the nutrient infusion device at a flow rate of 700 mL/h when the type A consumable is located within the roller structure, multiple test currents under type A consumables may be obtained. Assume that the average value of the test currents is measured to be 130 mA. The standard current range under type A consumables might then be (130×95% mA, 130×105% mA).

For example, for type B consumable, by conducting multiple tests on the nutrient infusion device at a flow rate of 700 mL/h when the type B consumable is located within the roller structure, multiple test currents under the type B consumable may be obtained, and the average value of the test currents might be calculated to obtain a current of 140 mA. The range of standard currents under the type B consumable could then be chosen to be (140×95% mA, 140×105% mA).

In some embodiments, the step of determining the state information of the medical device according to the operating state parameter of the drive member and the predetermined judgment rule includes the following steps.

The operating current of the drive member is compared with the standard current range for each consumable type, and the consumable type is output when the operating current of the drive member is within the standard current range for the consumable type.

In some embodiments, the step of determining the state information of the medical device according to the operating state parameter of the drive member and the predetermined judgment rule specifically includes the steps.

The operating current of the drive member is compared with the standard current range of the target consumable type, and the consumable type abnormal information is output when the operating current of the drive member is not within the standard current range of the target consumable type.

The target consumable type may be input by the user when using the nutrient infusion device, or it may be set automatically for when the nutrient infusion device enters a certain mode.

In the above technical solution, the tension force of the consumables may vary from one consumable type to another, and the corresponding operating current of the drive member may also be different. The working current of the drive member can be measured and compared with the standard current range of a certain consumable type, so as to identify the consumable type or to determine if the consumable type is being used incorrectly.

### Embodiment 13

Some embodiments of the invention provide a detection method which is applied to a medical device. The method comprises the following steps.

S1301, an operating state parameter of the drive member is acquired.

The operating state parameter of the drive member includes a torque value of the drive member. The torque value of the drive member when the medical device is running with a load is determined.

For example, the torque value of the drive member when the nutrient infusion device is running with a roller structure installed and consumables installed in the roller structure may be determined.

S1302, the state information of the medical device is determined according to the operating state parameter of the drive member and the predetermined judgment rule.

The predetermined judgment rule includes standard torque value ranges for a plurality of consumable types. The tension force of the consumables varies for different consumable types, and the corresponding torque values of the drive member are also different.

For each type of consumable, the range of standard torque values for various types of consumables is obtained by conducting multiple tests of the nutrient infusion device at a certain test flow rate when the consumable is located within the roller structure.

For example, for type A consumables, when the type A consumables are located within the roller structure, by conducting multiple tests on the nutrient infusion device at a flow rate of 700 mL/h, multiple test torque values under type A consumables are obtained, and the average of the test torque values is calculated to obtain a torque value of 117 mN·m. Then the standard torque value range under type A consumables is (117×95% mN·m, 117×105% mN·m).

For example, for type B consumables, when the type B consumable is mounted in the roller structure, by conducting multiple tests on the nutrient infusion device at a flow rate of 700 mL/h, multiple test torque values under type B consumables are determined, and the average of the test torque values may be calculated to obtain a torque value of, for example, 126 Nm. Then the standard torque value range under type B consumables is (126×95% Nm, 126×105% Nm).

In some embodiments, the step of determining the state information of the medical device according to the operating state parameter of the drive member and the predetermined judgment rule includes the following steps.

The torque value of the drive member is compared with a standard torque value range for each consumable type, and the consumable type is output when the torque value of the drive member lies within the standard torque value range for a consumable type.

In some embodiments, the step of determining the state information of the medical device according to the operating state parameter of the drive member and the predetermined judgment rule includes the following steps.

The torque value of the drive member is compared with a standard torque value range of the target consumable type, and the consumable type abnormal information is output when the torque value of the drive member is not within the standard torque value range of the target consumable type.

The target consumable type may be input by the user when using the nutrient infusion device, or it may be set automatically when the nutrient infusion device enters a certain mode.

In the above technical solution, the tension forces for different consumable types vary and correspond to different torque values for the drive member are measured. The torque value of the drive member is detected and the torque value of the drive member is compared with the range of the standard torque value of a certain consumable type, so as to identify the consumable type or to determine if the consumable type is being used incorrectly.

### Embodiment 14

Some embodiments of the invention provide a detection method, which is applied to a medical device. This method comprise the following steps.

S1401, an operating state parameter of the drive member is acquired.

The operating state parameter of the drive member includes an operating current of the drive member. The operating current of the drive member when the medical device is running with a load is measured.

For example, the working current of the drive member when the nutrient infusion device is running with a roller structure installed and consumables installed in the roller structure may be measured.

S1402, the state information of the medical device is determined according to the operating state parameter of the drive member and the predetermined judgment rule.

The predetermined judgment rules may include a consumable consistency evaluation current range. The length of the consumables affects the accuracy of the medical device, and different consumable lengths correspond to different operating currents of the drive member.

A plurality of currents in obtained by installing standard length consumables in the roller structure and conducting multiple tests of the medical device, and the plurality of currents are statistically analyzed to obtain the consumable consistency evaluation current range.

For example, with standard consumables are installed on the roller structure, the nutrient infusion device may be allowed to perform the liquid infusion test at a flow rate of 700 mL/h. The test currents of multiple motors are obtained, the average value of the test currents of multiple motors is calculated to be 125 mA, and the consumable consistency evaluation current range is set to be (125 mA-125×0.02 mA, 125 mA+125×0.02 mA).

The operating current of the driver is compared with the consumable consistency evaluation current range. When the operating current of the drive member is not within the consumable consistency evaluation current range, a length abnormality message of the consumables is output.

In the above technical solution, different consumable lengths correspond to different working currents of the drive member. The working current of the drive member is detected and the working current of the drive member is compared with the consumable consistency evaluation current range, so that whether or not the consumable length is abnormal is determined.

### Embodiments 15

Some embodiments of the invention provide a detection method which is applied to a medical device. The method specifically includes the following steps.

S1501, an operating state parameter of the drive member is acquired.

The operating state parameter of the drive member includes a torque value of the drive member. The torque value of the drive member when the medical device is running with load is acquired.

For example, the torque value of the drive member when the nutrient infusion device is running with a roller structure installed and consumables installed in the roller structure may be acquired.

S1502, the state information of the medical device is determined according to the operating state parameter of the drive member and the predetermined judgment rule.

The predetermined judgment rule includes a consumable consistency evaluation torque range. The length of consumables affects the accuracy of the medical device, and different consumable lengths correspond to different torque values of the drive member.

A plurality of torque values is obtained by installing standard-length consumables in the roller structure and conducting multiple tests of the medical device, and the plurality of torque values may be statistically analyzed to obtain the consumable consistency evaluation torque range.

For example, the standard consumables might be installed on the roller structure and the nutrient infusion device allowed to perform the liquid infusion test at a flow rate of 700 mL/h. The test torque values of multiple motors may then be measured, and the average value of the test torque values of multiple motors might be calculated to be 112.5 Nm. The range of consistency evaluation torque values of consumables is set to be (112.5 Nm-112.5×0.02 Nm, 112.5 Nm+112.5×0.02 Nm).

The torque value of the drive member is compared with the consumable consistency evaluation torque range. When the torque value of the drive member is not within the consumable consistency evaluation torque range, a consumable length abnormality information signal is output.

In the above technical solution, different consumable lengths correspond to different torque values of the drive member. The torque value of the drive member is detected and the torque value of the drive member is compared with the consumable consistency evaluation torque range, so that whether or not the consumable length is abnormal is determined.

### Embodiment 16

Some embodiments of the invention provide a detection method, which is applied to a medical device. The method comprises the following steps.

S1601, an operating state parameter of the drive member is determined.

The operating state parameter of the drive member includes an operating current of the drive member. The operating current of the drive member when the medical device is running with no load is measured.

For example, the working current of the drive member when the nutrient infusion device is running without the roller structure installed may be measured.

S1602, the state information of the medical device is determined according to the operating state parameter of the drive member and the predetermined judgment rule.

The predetermined judgment rule includes a no-load current threshold. The no-load current threshold is configured to detect whether the medical device works properly during no-load operation.

For example, the nutrient infusion device is allowed to operate at a flow rate of 700 mL/h, corresponding to the rotation speed with no load. After several tests, the test currents of multiple motors may be measured. The average value of the test currents of multiple motors might then be calculated as 80 mA, such that the no-load current threshold could be set to (80 mA+80×0.02 mA).

Whether the operating current of the driver is less than the no-load current threshold is determined. If the operating current is less than the no-load current threshold, then this indicates that the device is running normally with no load. The information that the device is running normally with no load can then be output. If the operating current is greater than or equal to within the no-load current threshold, then this indicates that the device is running abnormally with no load, and then information that the device is running abnormally, with no load, may be output.

In the above technical solution, the working current of the drive member is detected and the working current of the drive member is compared with the no-load current threshold, so that whether the medical device is abnormal in no-load operation is determined.

### Embodiment 17

Some embodiments of the invention provide a detection method which is applied to a medical device. The method comprises the following steps.

S1701, an operating state parameter of the drive member is acquired.

The operating state parameter of the drive member includes a torque value of the drive member. The torque value of the drive member when the medical device is running at no load is acquired.

For example, the torque value of the drive member when the nutrient infusion device is running without the roller structure installed is acquired.

S1702, the state information of the medical device is determined according to the operating state parameter of the drive member and the predetermined judgment rule.

The predetermined judgment rule includes a no-load torque value threshold. The no-load torque value threshold is used to detect whether the medical device works normally during no-load operation.

For example, the nutrient infusion device is allowed to rotate at a flow rate corresponding to a rotation speed of 700 mL/h with no load. After multiple tests, a test torque value of a plurality of motors is obtained, and an average of the test torque values of the plurality of motors is calculated to be 72 Nm. The no-load torque value threshold is set to be 72×1.05 Nm. The flow rate and the rotation speed satisfy the formula Q=0.25×n, where Q is the flow rate, and n is the rotation speed.

If the torque value is less than the no-load torque value threshold, this may indicate that the device is operating normally with no load, and then the information that the device is operating normally with no load is output. If the torque value is greater than or equal to within the no-load torque value threshold, it is indicated that the device is operating abnormally with no load, and then the information that the device is operating abnormally with no load information is output.

In the above-described technical solution, the torque value of the drive member is detected and the torque value of the drive member is compared with the no-load torque value threshold, so that whether the medical device is abnormal in no-load operation is determined.

It is to be noted herein that the embodiments involved in each of the above embodiments are combinable with each other.

Some embodiments of the invention provide a medical device including a controller, a drive member, and a drive control member. The controller is configured to control the drive control member to drive the drive member to operate, and the controller is configured to realize the detection method involved in the above embodiments.

An embodiment of the invention provides a controller, in which the controller includes a memory and a processor.

The memory is configured to store computer instructions executable by the processor;

The processor is configured to realize the various steps in the method in the foregoing embodiment when executing the computer instructions. Specific reference may be made to the relevant description in the foregoing embodiment of the method.

In some embodiments, the aforementioned memory may be either stand-alone or integrated with the processor. When the memory is stand-alone, the controller further includes a bus for connecting the memory to the processor.

Embodiments of the invention also provide a computer-readable storage medium having computer instructions stored therein. The processor is configured to execute the computer instructions to realize the various steps in the method in the above embodiment.

Embodiments of the invention also provide a computer program product including computer instructions. The computer instructions when executed by the processor, realizes the various steps in the method in the above embodiments.

Other embodiments of the invention will readily come to mind to those skilled in the art upon consideration of the specification and practice of the invention disclosed herein. The invention is intended to cover any variations, uses, or adaptations of the invention that follow the general principles of the invention and include common knowledge or customary technical means in the art not disclosed herein. The specification and embodiments are to be regarded as exemplary only, and the true scope and spirit of this invention is indicated by the following claims.

It is to be understood that this invention is not limited to the precise structure which has been described above and illustrated in the accompanying drawings, and that various modifications and changes may be made without departing from its scope. The scope of this invention is limited only by the appended claims.

1. A detection method applied to a medical device, the medical device comprising a controller, a drive member, and a drive control member; the controller being configured to control the drive control member to drive the drive member to operate, the method comprising:
acquiring an operating state parameter of the drive member;
determining state information of the medical device according to the operating state parameter of the drive member and a predetermined judgment rule; and
generating for a user an indication of at least one of a normal state or abnormal state of the medical device depending on the determined state information.

2. The method of claim 1, wherein the operating state parameter is related to a load driven by the drive member.

3. The method of claim 1, wherein the operating state parameter comprises at least one of the following: a voltage value, a current value, a torque, and a rotation speed.

4. The method of claim 1, further comprising outputting the state information.

5. The method of claim 1, wherein the medical device includes a force sensor, the force sensor being placed at a load end to which the drive member is connected;
the step of acquiring an operating state parameter of the drive member comprising:
acquiring a torque measurement voltage from the force sensor; and
obtaining a torque value of the drive member according to a torque measurement voltage and a preset torque conversion factor.

6. The method of claim 5, wherein the step of obtaining the torque value of the drive member according to the torque measurement voltage and the preset torque conversion factor comprises:
determining a product of the torque measurement voltage and the predetermined torque conversion factor as the torque value of the drive member.

7. The method of claim 1, wherein
the medical device is a nutrient infusion device, and
the nutrient infusion device further comprises a speed reducer and a roller structure;
the drive member is drivingly connected to the speed reducer, and
the speed reducer is drivingly connected to the roller structure;
wherein
the roller structure is configured to rotate under the influence of the drive member and to drive the flow of a fluid in consumables; and
the roller structure is removably connected to the speed reducer by a quick release coupling clasp.

8. The method of claim 7, wherein the step of determining the state information of the medical device according to the operating state parameters of the drive member and the predetermined judgment rule comprises:
comparing an operating current of the drive member with a range of consumable dislodgement current corresponding to the rotation speed of the drive member during a period of use of the consumables;
outputting an alarm message for dislodgement of the consumables when the operating current of the drive member is within the range of a consumable dislodgement current.

9. The method of claim 8, wherein the nutrient solution infusion apparatus further comprises an in-position detection member, the in-position detection member being disposed opposite the roller structure, the method further comprising
the step of outputting the alarm message indicating dislodgement of the consumable when the operating current of the drive member is within the range of the consumable dislodgement current comprises:
outputting the alarm message indicating dislodgement of the consumable when the operating current of the drive member is within the range of the consumable dislodgement current and the in-position detection member detects that the roller structure is in position.

10. The method of claim 9, wherein the method further comprises:
outputting a roller structure in-position exception message when the operating current of the drive member is within a range of the consumable dislodgement current and the in-position detection member detects that the roller structure is not in position.

11. A medical device, comprising: a controller, a drive member, a drive control member; wherein the controller is configured to control the drive control member to drive the drive member to operate, and further configured to:
acquire an operating state parameter of the drive member; and
determine state information of the medical device according to the operating state parameter of the drive member and a predetermined judgment rule.

12. The medical device of claim 11, wherein the operating state parameter is related to a load driven by the drive member.

13. The medical device of claim 11, wherein the operating state parameter comprises at least one of the following: a voltage value, a current value, a torque, and a rotation speed.

14. The medical device of claim 11, wherein the controller is further configured to:
output the state information.

15. The medical device of claim 11, further comprising a force sensor, the force sensor being placed at a load end to which the drive member is connected;
wherein the controller acquires an operating state parameter of the drive member by:
acquiring a torque measurement voltage from the force sensor; and
obtaining a torque value of the drive member according to a torque measurement voltage and a preset torque conversion factor.

16. The medical device of claim 12, wherein the controller obtains the torque value of the drive member according to the torque measurement voltage and the preset torque conversion factor by:
determining a product of the torque measurement voltage and the predetermined torque conversion factor as the torque value of the drive member.

17. The medical device of claim 11, wherein the medical device is a nutrient infusion device, and the nutrient infusion device further comprises a speed reducer and a roller structure; the drive member is drivingly connected to the speed reducer, and the speed reducer is drivingly connected to the roller structure; wherein the roller structure is configured to rotate under the drive of the drive member and to drive the flow of a fluid in consumables; and the roller structure is removably connected to the speed reducer by a quick release coupling clasp.

18. A computer-readable storage medium having computer instructions stored therein, wherein the computer instructions when executed by a processor are configured to:
acquire an operating state parameter of the drive member; and
determine state information of the medical device according to the operating state parameter of the drive member and a predetermined judgment rule.
